(19) United States
(12) Patent Application Publication
(10) Pub. No.: US 2014/0335121 A1
(43) Pub. Date: Nov. 13, 2014

(54) FIMBRIAE TYPE OF PROPHYROMONAS GULAE

(71) Applicants: School Corporation, Azabu Veterinary Medicine Educational Institution, Sagamihara-shi, Kanagawa (JP); Osaka University, Suita-shi, Osaka (JP)

(72) Inventors: Fumitoshi Asai, Kanagawa (JP); Yukio Kato, Kanagawa (JP); Mitsuyuki Shirai, Kanagawa (JP); Masaru Murakami, Kanagawa (JP); Kazuhiko Nakano, Osaka (JP); Ryota Nomura, Osaka (JP)

(21) Appl. No.: 14/364,810

(22) PCT Filed: Dec. 13, 2012
(86) PCT No.: PCT/JP2012/082288

§ 371 Date: Jun. 12, 2014

(30) Foreign Application Priority Data
Dec. 13, 2011 (JP) 2011-272679

(51) Int. Cl. C07K 14/195; C12Q 1/68

(52) U.S. Cl. CPC C07K 14/195; C12Q 1/689
USPC 424/190.1; 530/350; 530/387.9; 435/6.12

(57) ABSTRACT

The present invention has as its object to provide a process for determining the degree of pathogenicity of infecting bacterial strains in the case of canine periodontal disease.

The present inventors found that Porphyromonas gulae is a major pathogen of canine periodontal disease, and demonstrated that the fimA genes encoding FimA (fimbrillin) protein which constitute fimbriae of this bacterium are classified into three major groups, and that each of the groups is correlated with the pathogenicity of this bacterium as a pathogenic bacterial of periodontal disease. Thus the inventors completed the present invention.

### TECHNICAL FIELD

The present invention relates to classification of the fimbrial types of Porphyromonas gulae, which is believed to be a pathogenic microorganism of canine periodontal disease. This invention also relates to prediction of the pathogenicity of the periodontal disease based on this classification of the fimbrial types.

### BACKGROUND ART

Periodontal disease is a generic name for diseases that occur in the periodontal tissues and destroy said tissues. It is known that the disease has very high prevalence among elderly humans in Japan.

Periodontal disease in animals, notably companion animals such as dogs and cats, is also known as an oral infectious disease with high prevalence. For example, it is said that in the United States, 85% of dogs and 75% of cats over three years old suffer from periodontal disease. However, unlike in humans, dental treatments in the oral cavity are not common in dogs and cats, so periodontal disease goes beyond just being an oral infectious disease and is a serious disease that may lead to the loss of multiple teeth and potentially even to the shortening of life as the pathology advances.

It is known that in the case of human periodontal disease, the pathogenicity of bacterial infection varies with the type of the infecting bacterium or the strain type of said bacterium (Non-patent Document 1). If the pathogenicity of pathogens of periodontal disease in animals such as dogs and cats can be determined, this will enable one to predict the risk of onset of this disease, as well as to choose an appropriate therapy for the symptom of an animal affected by this disease in the process of designing a treatment strategy for the animal. However, there has been no known process for determining the degree of pathogenicity of pathogens of periodontal disease in animals.

### PRIOR ART DOCUMENTS

### Non-Patent Documents

Non-patent Document 1: Nakano K., et al., Oral Microbiol. Immunol., 2004, 19, 205-209

### SUMMARY OF THE INVENTION

### Problems to be Solved by the Invention

The present invention has as its object to provide a process for determining the degree of pathogenicity of infecting bacterial strains in the case of canine periodontal disease.

### Means for Solving the Problems

The present inventors demonstrated that with regard to Porphyromonas gulae, which is believed to be a major pathogen of canine periodontal disease, the fimA genes encoding FimA (fimbrillin) protein which constitute fimbriae of this bacterium are classified into three major groups, and that each of the groups is correlated with the pathogenicity of this bacterium as a pathogenic bacterium of periodontal disease. Thus, the inventors completed the present invention.

More specifically, the present inventors analyzed the amino acid sequences of FimA (fimbrillin) protein from Porphyromonas gulae strains by the neighbor-joining method in comparison with the amino acid sequences of FimA protein from the strains of Porphyromonas gingivalis known as a major pathogenic bacterium of human periodontal disease and, as a result, classified the amino acid sequences of the Porphyromonas gulae strains into two groups (groups B and C) consisting of those sequences determined to be similar to the amino acid sequences of Porphyromonas gingivalis FimA protein and the other group (group A) consisting of those sequences specific to Porphyromonas gulae. The inventors also investigated these groups using a murine peritoneal model reflecting the degree of periodontal pathogenicity and, as a result, found that groups A, B and C have low, medium, and high pathogenicities, respectively.

On the basis of the comparison of the amino acid sequences of FimA protein from these groups with those of Porphyromonas gingivalis strains, regions characteristic of the protein from the respective groups or the DNAs encoding the same were identified. It was found that among these groups, identification of the amino acid sequence of a partial peptide characteristic of FimA protein from group C with high pathogenicity makes it possible to provide the characteristic partial peptide per se, proteins comprising the partial peptide (e.g., protein with SEQ ID NO: 6), and antibodies against such proteins, notably antibodies that bind to the partial peptide characteristic of FimA protein from group C or the proteins comprising said characteristic partial peptide but not to FimA protein of the other groups (i.e., group A or B). The inventors also found that the characteristic partial peptide or the proteins comprising the partial peptide as mentioned above can be used in the living body as a vaccine for eliciting an immune against FimA protein from Porphyromonas gulae strains of group C, and that the antibodies against the partial peptide or the proteins can be used as therapeutic periodontal disease drugs for use in the oral cavity.

Further, through the use of the gene sequences specific to the respective group, the inventors constructed a process for identifying the presence and FimA groups of Porphyromonas gulae strains on the basis of analysis of dental plaques collected from the oral cavities of dogs. Thus, the inventors have come to establish determination of the risk of periodontal disease in a dog using its dental plaque.

### Advantageous Effects of the Invention

The process of the present invention enables determination of the degree of risk of onset of periodontal disease in dogs by classifying FimA (fimbrillin) protein from strains of Porphyromonas gulae, a major pathogen for periodontal disease infection in dogs, into three groups A to Con the basis of the amino acid sequences of said proteins. Further, the results of pathogenicity determination can he relied upon to choose a therapy for canine periodontal disease depending on the degree of pathogenicity of an infecting bacterium. The results can also serve as a measure of whether the therapy takes effect.

### BRIEF DESCRIPTIONS OF THE DRAWINGS

FIG. 1 shows the alignment of FimA protein from ATCC51700, D044 and D049 which are representative bacterial strains having FimA protein classified as groups A, B and C, respectively.

FIG. 2 shows a phylogenetic tree comparing the putative amino acid sequences (SEQ ID NO: 2) of FimA (fimbrillin) protein from Porphyromonas gulae (indicated as “Pgu” in the figure) with those from Porphyromonas gingivalis (indicated as “Pgi” in the figure).

### MODES FOR CARRYING OUT THE INVENTION

The present inventors searched for pathogenic bacteria of periodontal disease in dogs and found that Porphyromonas gulae is a major pathogen of periodontal disease in dogs (Kato Y., et al., J. Vet. Dent., vol. 28, no. 2, Summer 2011, p. 84-89). There are known bacteria considered to cause periodontal disease in humans, such as Porphyromonas gingivalis, Tannerella forsythia, and Campylobacter rectus which have high pathogenicity. Porphyromonas gulae which was found this time to be normally present in the oral cavities of dogs is a bacterium belonging to the same genus as Porphyromonas gingivalis which is considered to be an important pathogen of periodontal disease in humans. However, the results of the investigation at this time showed that Porphyromonas gingivalis is only present in very limited numbers in the oral cavities of dogs; so periodontal diseases in humans and dogs were considered to be caused by different mechanisms.

As to Porphyromonas gingivalis, it has been known that the pathogenicity of periodontal disease in humans varies greatly with the genotype of the fimA gene specifying FimA (fimbrillin) protein which constitutes fimbriae. It has also been known that the fimA genotypes of Porphyromonas gingivalis are classified into six groups: types I, Ib, II, III, IV and V, and that among these groups, types II, IV and Ib are genotypes with high pathogenicity which are isolated with high frequency from periodontal disease patients while types I, III and V are genotypes with low pathogenicity which are isolated with high frequency from non-periodontal disease patients.

Considering that Porphyromonas gulae strains isolated from the oral cavities of dogs also carry fimbrial FimA protein, the present inventors further analyzed in detail the sequences of the fimA genes encoding FimA protein from the bacterial strains. As a result, the inventors demonstrated that Porphyromonas gulae FimA protein are classified into three groups (groups A, B and C), and that each of the groups is correlated with the pathogenicity of the bacterial strains viewed as pathogens of periodontal disease. Thus, the inventors completed the present invention.

Here, among the Porphyromonas gulae strains studied herein, examples of the bacterial strains having FimA protein classified as group A include, but are not limited to, D024, D025, D028, D034, D035, D036, D042, D043, D060, D066, D067, D068, and ATCC51700. Examples of the bacterial strains having FimA protein classified as group B include, but are not limited to, D040, D044, D052, D053, D077, and B43. Examples of the bacterial strains having FimA protein classified as group C include, but are not limited to, D049.

More specifically, the present inventors analyzed the amino acid sequences of FimA (fimbrillin) protein from Porphyromonas gulae strains by the neighbor-joining method in comparison with the amino acid sequences of FimA protein from Porphyromonas gingivalis strains and. as a result, classified the amino acid sequences of the Porphyromonas gulae strains into two groups (groups B and C) consisting of those sequences determined to be similar to the amino acid sequences of Porphyromonas gingivalis FimA protein and the other group (group A) consisting of those sequences specific to Porphyromonas gulae. The inventors also investigated these groups using a murine peritoneal model and, as a result, found that groups A, B and C have low, medium, and high pathogenicities, respectively. Further, through the use of the gene sequences specific to the respective groups, the inventors constructed a process for identifying the presence and FimA groups of Porphyromonas gulae strains on the basis of analysis of dental plaques collected from the oral cavities of dogs. Thus, the inventors have come to establish determination of the risk of periodontal disease in a dog using its dental plaque.

The strain selected as a representative Porphyromonas gulae strain classified as group A is ATCC51700; and the nucleotide sequence of the fimA gene from this strain, and the amino acid sequence of the FimA protein encoded by said gene, are shown in SEQ ID NOs: 1 and 2, respectively. The strain selected as a representative Porphyromonas gulae strain classified as group B is D044; and the nucleotide sequence of the fimA gene from this strain, and the amino acid sequence of the FimA protein encoded by said gene, are shown in SEQ ID NOs: 3 and 4, respectively. The strain selected as a representative Porphyromonas gulae strain classified as group C is D049; and the nucleotide sequence of the fimA gene from this strain, and the amino acid sequence of the FimA protein encoded by said gene, are shown in SEQ ID NOs: 5 and 6, respectively.

In this method, the FimA protein from certain Porphyromonas gulae strains isolated from dogs are compared at the amino acid sequence level with the FimA protein from Porphyromonas gingivalis strains. In this case, the nucleotide sequences of the fimA genes encoding the FimA protein are first sequenced from the above-mentioned certain Porphyromonas gulae strains, and the amino acid sequences of the FimA protein from said Porphyromonas gulae strains are specified on the basis of said nucleotide sequences. Then, a phylogenetic tree is constructed by analyzing said amino acid sequences by the neighbor-joining method in comparison with the known amino acid sequences of some Porphyromonas gingivalis FimA protein, whereby the amino acid sequences of the Porphyromonas gulae FimA protein are classified.

The results of this analysis showed that the amino acid sequences of the Porphyromonas gulae FimA protein are classified into three groups A to C. Group A is a group having a sequence specific to Porphyromonas gulae; group B is a group having a sequence similar to that of the low-pathogenicity Porphyromonas gingivalis group, type III; and group C is a group having a sequence similar to that of the high-pathogenicity Porphyromonas gingivalis group, type IV.

In the present invention, further investigation was made using ATCCS 1700 as a standard strain of Porphyromonas gulae. As a result, it was found that the bacterial strains of groups A, B and C have low, somewhat high and very high pathogenicities in an animal model (i.e., murine abscess model), respectively. The results are summarized in Table 1.

TABLE 1
Summary of FimA protein from Porphyromonas gingivalis strains

| | Group A | Group B | Group C |
|---|---|---|---|
| Number of strains sequenced | 13 | 6 | 1 |
| Total length (bp) | 1155 | 1164 | 1167 |
| Pathogenicity in animal model | Low | Somewhat high | High |
| Periodontal pathogenicity | Low | Somewhat high | High |

The present invention provides a simpler process for identifying the groups of Porphyromonas gulae strains on the basis of these embodiments. To be specific, dental plaque samples are collected from dogs, and bacterial DNAs present in the samples are extracted. With the Porphyromonas gulae DNAs present in the bacterial DNAs being used as templates, initial screening is performed using a Porphyromonas gulae-specific primer set (5′-ttg ctt ggt tgc atg atc gg-3′ (SEQ ID NO: 7) and 5′-gct tat tct tac ggt aca ttc aca-3′ (SEQ ID NO: 8)) to thereby detect Porphyromonas gulae strains. The samples identified as positive in the initial screening are further subjected to PCR using the following primer set: a primer set that is specific to fimbrial fimA genes from Porphyromonas gulae strains of group A (5′-tga gaa tat caa atg tgg tgc agg ctc acg-3′ (SEQ ID NO: 9) and 5′-ctt gcc tgc ctt caa aac gat tgc ttt tgg-3′ (SEQ ID NO: 10)); a primer set specific to fimbrial fimA genes from Porphyromonas gulae strains of group B (5′-taa gat tga agt gaa gat gag cga ttc tta tgt-3′ (SEQ ID NO: 11) and 5′-att tcc tca gaa ctc aaa gga gta cca tca-3′ (SEQ ID NO: 12)); and a primer set that is specific to fimbrial fimA genes from Porphyromonas gulae strains of group C ([5′-cga tta tga cct tgt cgg taa gag ctt gga-3′ (SEQ ID NO: 13) and 5′-tgt ggc ttc gtt gtc gca gaa tcc ggc atg-3′ (SEQ ID NO: 14)] or [5′-gat ttg ctg ctc ttg cta tga cag ctt gta-3′ (SEQ ID NO: 19) and 5′-ttt agt cgt ttg acg ggt cga tta cca agt-3′ (SEQ ID NO: 20)]); whereby fimbrial types are detected. This process enables the grouping of Porphyromonas gulae strains using PCR without performing the heavy-duty work of constructing a phylogenetic tree through the comparison between the amino acid sequences of complex proteins.

On the basis of these findings, the present invention can also provide a kit for identifying the groups of Porphyromonas gulae strains, which enables classification of the amino acid sequences of Porphyromonas gulae FimA (fimbrillin) protein into three groups A to C.

In such a kit, there can be used, for example, primer sets for classifying the amino acid sequences of Porphyromonas gulae FimA (fimbrillin) protein into three groups A to C.

For example, the kit can comprise the primer sets:

a primer set that is specific to fimbrial fimA genes from Porphyromonas gulae strains of group A [5′-tga gaa tat caa atg tgg tgc agg ctc acg-3′ (SEQ ID NO: 9) and 5′-ctt gee tgc ctt caa aac gat tgc ttt tgg-3′ (SEQ ID NO: 10)] or [5′-ttc ata cgt cga cga ctg cg-3′ (SEQ ID NO: 15) and 5′-ttg agg gtt gat tac caa gt-3′ (SEQ ID NO: 16)];

a primer set that is specific to fimbrial fimA genes from Porphyromonas gulae strains of group B [5′-taa gat tga agt gaa gat gag cga ttc tta tgt-3′ (SEQ ID NO: 11) and 5′ att tcc tca gaa ctc aaa gga gta cca tca-3′ (SEQ ID NO: 12)] or [5′-aac tac gac gct ata tgc aa-3′ (SEQ ID NO: 17) and 5′-tag aca aac tat gaa agt t-3′ (SEQ ID NO: 18)]; and

a primer set that is specific to fimbrial fimA genes from Porphyromonas gulae strains of group C [5′-cga tta tga cct tgt cgg taa gag ctt gga-3′ (SEQ ID NO: 13) and 5′-tgt ggc ttc gtt gtc gca gaa tcc ggc atg-3′ (SEQ ID NO: 14)] or [5′-gat ttg ctg ctc ttg cta tga cag ctt gta-3′ (SEQ ID NO: 19) and 5′-ttt agt cgt ttg acg ggt cga tta cca agt-3′ (SEQ ID NO: 20)]. However, those skilled in the art reading the disclosures in the present specification could naturally understand that the primer sets are not limited to the above-mentioned ones, and could also select other primer sets as appropriate making reference to the sequences of the fimbrial fimA genes from Porphyromonas gulae strains of the three groups A to C.

On the basis of these findings, the present invention can further provide a partial peptide of FimA (fimbrillin) protein from Porphyromonas gulae strains classified as group C, which has an amino acid sequence (SEQ ID NO: 2) specified by the nucleotide sequence moiety of SEQ ID NO: 21 which is amplified using a primer set that is specific to fimbrial fimA genes from Porphyromonas gulae strains of group C [5′-cga tta tga cct tgt cgg taa gag ctt gga-3′ (SEQ ID NO: 13) and 5′-tgt ggc ttc gtt gtc gca gaa tcc ggc atg-3′ (SEQ ID NO: 14)], as well as a protein comprising said partial peptide (for example, a protein having the amino acid sequence of SEQ ID NO: 6). The partial peptide, which is a sequence characteristic of the FimA protein from group C as compared with the FimA protein from the other groups (i.e., group A or B), was expected to have a possible relationship with very high pathogenicity of the Porphyromonas gulae strains of group C.

By producing, in the living body, an antibody specifically binding to said partial peptide (i.e., an antibody that binds to said partial peptide or said protein comprising said partial peptide (e.g., a protein having the amino acid sequence of SEQ ID NO: 6) but not to the FimA protein from the other groups (i.e., group A or B)), or by extrinsically applying the antibody having such binding properties in the form of intraoral spray, gel, or the like, animal periodontal disease or associated diseases involved by the Porphyromonas gulae strains of group C can be treated.

As one embodiment of this aspect of the present invention, there can also be provided a vaccine against FimA (fimbrillin) protein from Porphyromonas gulae strains classified as group C, which comprises, as an immunogen, said partial peptide or the protein comprising said partial peptide.

### EXAMPLES

### Example 1

### Analysis of FimA Protein from Porphyromonas gulae Strains

In this example, the amino acid sequences of FimA protein were attempted to be specified from different Porphyromonas gulae strains isolated from the oral cavities of dogs.

First, Porphyromonas gulae strains were obtained by collecting dental plaque samples from the oral cavities of 20 canine individuals. The dental plaque samples from these canine individuals were collected from the surface of teeth in the respective individuals using scalers. The collected dental plaque samples were dispersed in sterilized distilled water in sterilized plastic tubes, and bacterial DNAs were extracted from the bacterial dispersions.

Next, with the resulting DNAs being used as templates, PCR was performed using the following primer set: a primer set that is specific to fimbrial fimA genes from Porphyromonas gulae strains of group A (5′-ttc ata cgt cga cga ctg cg-3′ (SEQ ID NO: 15) and 5′-ttg agg gtt gat tac caa gt-3′ (SEQ ID NO: 16)); and a primer set that is specific to fimbrial fimA genes from Porphyromonas gulae strains of group B (5′-aac tac gac gct ata tgc aa-3′ (SEQ ID NO: 17) and 5′-tag aca aac tat gaa agt t-3′ (SEQ ID NO: 18)); a primer set that is specific to fimbrial fimA genes from Porphyromonas gulae strains of group C (5′-gat ttg ctg ctc ttg cta tga cag ctt gta-3′ (SEQ ID NO: 19) and 5′-ttt agt cgt ttg acg ggt cga tta cca agt-3′ (SEQ ID NO: 20)). The PCR conditions were as follows: initial denaturation at 95° C. for 5 minutes, followed by 30 cycles of a cycle consisting of 94° C. for 30 seconds, 62° C. for 30 seconds, and 72° C. for 30 seconds, and final extension at 72° C. for 5 minutes.

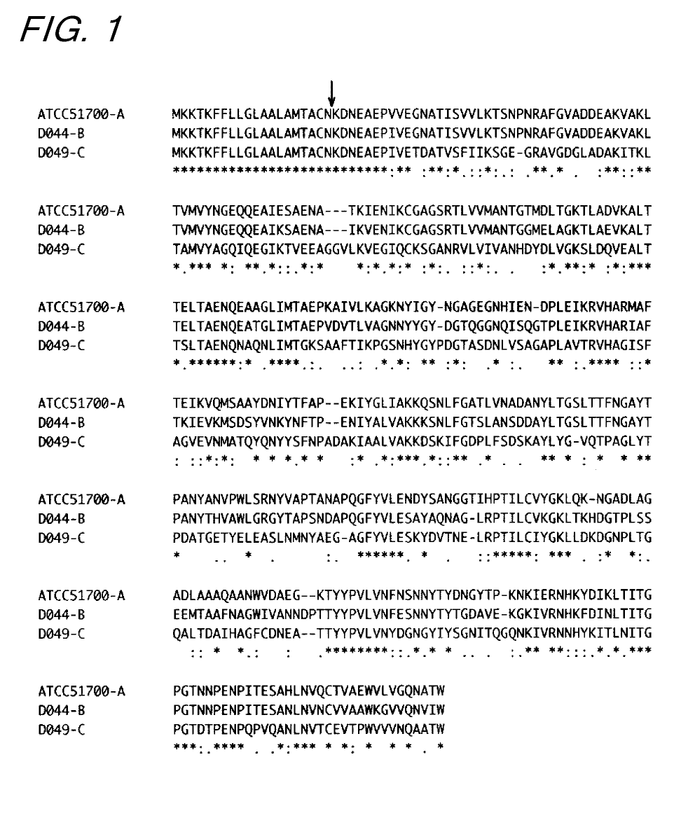

The nucleotide sequences of the fimA genes from the Porphyromonas gulae strains amplified via the above-mentioned PCR reaction were sequenced. As the result of the analysis, the nucleotide sequences of 1155 bp were obtained from 13 bacterial strains (D024, D025, D028, D034, D035, D036, D042, D043, D060, D066, D067, D068, and ATCC 51700), the nucleotide sequences of 1164 by from 6 strains (D040, D044, D052, D053, D077, and B43), and the nucleotide sequence of 1167 by from one strain (D049), with the respective groups being classified as groups A, B and C. As representative sequences from the respective groups, the nucleotide sequence of the fimA gene from ATCC51700, a bacterial strain belonging to group A. is shown in SEQ ID NO: 1, the nucleotide sequence of the fimA gene from D044, a bacterial strain belonging to group B, is shown in SEQ ID NO: 3, and the nucleotide sequence of the fimA gene from D049, a bacterial strain belonging to group C, is shown in SEQ ID NO: 5. In order to demonstrate the similarity among the sequences of the FimA protein of the three groups A to C, an alignment was constructed with these sequences (FIG. 1).

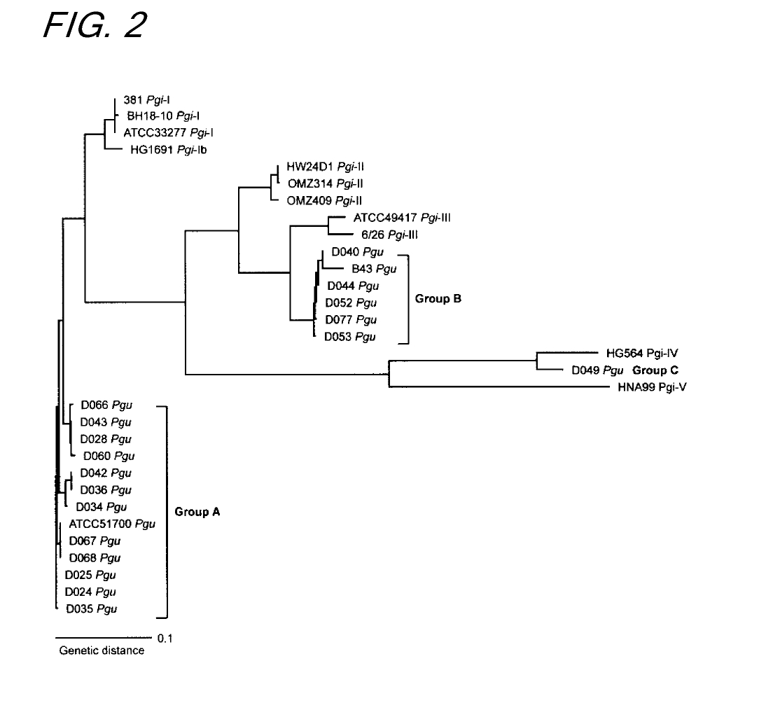

Thereafter, the amino acid sequences (SEQ ID NO: 2, SEQ ID NO: 4, and SEQ ID NO: 6, respectively) encoded by the resulting nucleotide sequences were compared with the nucleotide sequences of the fimA genes from Porphyromonas gingivalis strains through analysis by the neighbor-joining method, whereby the amino acid sequences of the Porphyromonas gulae FimA protein were classified. The results of the classification are shown in FIG. 2. In this figure, the abbreviation “Pgu” stands for a Porphyromonas gulae protein. In this phylogenetic tree, the similar amino acid sequences (e.g., SEQ ID NO: 2) obtained from 13 bacterial strains were found to be relatively different from the amino acid sequences of the Porphyromonas gingivalis FimA protein and to constitute a unique group (this group is referred to as group A). The similar amino acid sequences (e.g., SEQ ID NO: 4) obtained from 6 bacterial strains were found to contain the amino acid sequences similar to those of the FimA protein from the low-pathogenicity Porphyromonas gingivalis group, type III (this group is referred to as group B). The amino acid sequence (SEQ ID NO: 6) obtained from one bacterial strain was found to contain the amino acid sequences similar to those of the FimA protein from the high-pathogenicity Porphyromonas gingivalis group, type IV (this group is referred to as group C).

### Example 2

### Evaluation of the Pathogenicity of Different Bacterial Strains in a Murine Abscess Model

In this example, different Porphyromonas gulae strains were subcutaneously injected into the backs of mice to investigate inflammatory changes caused by the strains. It has been previously reported that the pathogenicity in this murine abscess model reflects periodontal pathogenicity (Nakano K., et al., Oral Microbiol. Immunol., 2004, 19, 205-209).

The obtained bacterial strains were serially grown in Gifu anaerobic medium (GAM) broth, and it was confirmed by PCR as mentioned in Example 1 that the resulting colonies were those of Porphyromonas gulae strains of interest. The grown strains were recovered and washed in a sterilized phosphate buffer solution (10 mM phosphate buffer solution containing 0.15 M sodium chloride; pH 7.4).

All animal tests were conducted according to the protocol approved by the Animal Research Committee of Osaka University Graduate School of Dentistry. Female BALB/c mice (5 weeks old) were raised with sterilized food and water ad libitum. 90 mice were divided into 9 groups of 10 mice each (8 test groups and 1 control group) to investigate inflammatory changes caused by subcutaneous injection of Porphyromonas gulae strains into the backs of the mice. The test groups used were those groups injected with any of the following Porphyromonas gulae strains: D049, D044, D040, ATCC51700, D035, D036, and D034. The negative control group used was the group injected with PBS instead of a bacterial strain, and the positive control group used was the group injected with OMZ314, a high-pathogenicity Porphyromonas gingivalis strain.

When the mice were 40 days old, they were each subcutaneously injected with 0.1 mL of a bacterial suspension (1×10⁹ colony forming units (CFU)) (for test groups) or 0.1 mL of the phosphate buffer solution (for control group) at a point 1 cm to the right of the midline of the back.

The mice were each monitored for signs of infection such as abscess formation and erosion formation as well as for spleen weight after infection. Spleen weight was measured two weeks after infection by euthanizing the mice under anesthesia and excising and weighing spleens. The results are summarized in Table 2.

TABLE 2
Pathogenicity evaluation

| Strain | Group | Observation during follow-up | Spleen weight |
|---|---|---|---|
| D049 | C | All mice died | — |
| OMZ314 | II | Abscess formed (9/10) | 5.06 ± 0.27 |
| D044 | B | Abscess formed (7/10) | 5.33 ± 0.28 |
| D040 | B | Abscess formed (10/10) | 5.04 ± 0.30 |
| ATCC51700 | A | — | 4.35 ± 0.14 |
| D035 | A | — | 4.26 ± 0.18 |
| D036 | A | — | 4.09 ± 0.10 |
| D034 | A | Abscess formed (1/10) | 3.67 ± 0.09 |
| PBS | — | — | 3.79 ± 0.20 |

The results demonstrated that whereas the bacterial strains of group A showed only very weak pathogenicity, the strains of group B caused abscess formation n many cases, and the strains of group C caused death in all animals.

### INDUSTRIAL APPLICABILITY

The process of the present invention enables determination of the degree of pathogenicity of periodontal disease in dogs by classifying FimA (fimbrillin) protein from strains of Porphyromonas gulae, a major pathogen for periodontal disease infection in dogs, into three major groups A to C on the basis of the amino acid sequences of said proteins. Further, the results of this pathogenicity determination can be relied upon to choose a therapy for canine periodontal disease depending on the degree of pathogenicity of an infecting bacterium.

### SEQUENCE LISTING FREE TEXT

SEQ ID NO: 1: Nucleotide sequence of the fimA gene encoding the FimA (fimbrillin) protein from ATCC51700 which is a reference Porphyromonas gulae strain (group A).
SEQ ID NO: 2: Amino acid sequence of the FimA protein encoded by the nucleotide sequence of SEQ ID NO: 1.
SEQ ID NO: 3: Nucleotide sequence of the fimA gene encoding the FimA protein from a Porphyromonas gulae strain of group B.
SEQ ID NO: 4: Amino acid sequence of the FimA protein encoded by the nucleotide sequence of SEQ ID NO: 3.
SEQ ID NO: 5: Nucleotide sequence of the fimA gene encoding the FimA protein from a Porphyromonas gulae strain of group C.
SEQ ID NO: 6: Amino acid sequence of the FimA protein encoded by the nucleotide sequence of SEQ ID NO: 5.
SEQ ID NO: 7 and SEQ ID NO: 8: Porphyromonas gulae-specific primer set.
SEQ ID NO: 9 and SEQ ID NO: 10: Primer pair for use to amplify the nucleotide sequence of the fimbrial fimA gene from a Porphyromonas gulae strain of group A.
SEQ ID NO: 11 and SEQ ID NO: 12: Primer pair for use to amplify the nucleotide sequence of the fimbrial fimA gene from a Porphyromonas gulae strain of group B.
SEQ ID NO: 13 and SEQ ID NO: 14: Primer pair for use to amplify the nucleotide sequence of the fimbrial fimA gene from a Porphyromonas gulae strain of group C.
SEQ ID NO: 15 and SEQ ID NO: 16: Primer pair for use to amplify the nucleotide sequence of the fimbrial fimA gene from a Porphyromonas gulae strain of group A.
SEQ ID NO: 17 and SEQ ID NO: 18: Primer pair for use to amplify the nucleotide sequence of the fimbrial fimA gene from a Porphyromonas gulae strain of group B.
SEQ ID NO: 19 and SEQ ID NO: 20: Primer pair for use to amplify the nucleotide sequence of the fimbrial fimA gene from a Porphyromonas gulae strain of group C.
SEQ ID NO: 21: Partial sequence of the nucleotide sequence of the fimbrial fimA gene from a Porphyromonas gulae strain of group C, which is amplified by the primer pair consisting of a combination of SEQ ID NO: 13 and SEQ ID NO: 14.
SEQ ID NO: 22: Partial peptide of the FimA (fimbrillin) protein from a Porphyromonas gulae of group C, which is contained in the partial nucleotide region of SEQ ID NO: 21.

1. A partial peptide of FimA (fimbrillin) protein from a Porphyromonas gulae strain classified as group C, which comprises the amino acid sequence of SEQ ID NO: 22.

2. The partial peptide according to claim 1, which is encoded by the nucleotide sequence of SEQ ID NO: 21.

3. A protein comprising the partial peptide according to claim 1.

4. The protein according to claim 3, comprising the amino acid sequence of SEQ ID NO: 6.

5. The protein according to claim 4, which is encoded by the nucleotide sequence of SEQ ID NO: 5.

6. An antibody which binds to the partial peptide according to claim 1 but not to a protein comprising the amino acid sequence of SEQ ID NO: 2 or 4.

7. The antibody according to claim 6, which is applied in the form of intraoral spray, gel, or the like.

8. A vaccine against FimA (fimbrillin) protein from a Porphyromonas gulae strain classified as group C, which comprises the partial peptide according to claim 1 as an immunogen.

9. A process for determining the degree of pathogenicity of Porphyromonas gulae strains in canine periodontal disease, which comprises the steps of:
classifying FimA (fimbrillin) protein from Porphyromonas gulae strains whose groups are unknown into three groups, i.e., groups A to C; and
determining the Porphyromonas gulae strains classified as groups B and C to be pathogenic groups.

10. The process according to claim 9, further comprising the step of determining the Porphyromonas gulae strains classified as group C to be highly pathogenic.

11. The process according to claim 9, wherein the amino acid sequences of the FimA protein are specified by sequencing the entire nucleotide sequences of genes encoding the FimA protein from the Porphyromonas gulae strains.

12. A process for identifying the groups of Porphyromonas gulae strains, which comprises the steps of
classifying the amino acid sequences of FimA (fimbrillin) protein from Porphyromonas gulae strains whose groups are unknown into three groups, i.e., groups A to C; and
determining the Porphyromonas gulae strains classified as groups B and C to be pathogenic groups.

13. The process according to claim 12, wherein the FimA protein from the Porphyromonas gulae strains are classified into groups A to C by the step of:
subjecting bacterial DNAs present in canine dental plaque samples to PCR using the following primer sets: a primer set that is specific to fimbrial fimA genes from Porphyromonas gulae strains of group A [5′-tga gaa tat caa atg tgg tgc agg ctc acg-3′ (SEQ ID NO: 9) and 5′-ctt gcc tgc ctt caa aac gat tgc ttt tgg-3′ (SEQ ID NO: 10)] or [5′-ttc ata cgt cga cga ctg cg-3′ (SEQ ID NO: 15) and 5′-ttg agg gtt gat tac caa gt-3′ (SEQ ID NO: 16)]; a primer set that is specific to fimbrial fimA genes from Porphyromonas gulae strains of group B [5′-taa gat tga agt gaa gat gag cga ttc tta tgt-3′ (SEQ ID NO: 11) and 5′-att tcc tca gaa ctc aaa gga gta cca tca-3′ (SEQ ID NO: 12)] or [5′-aac tac gac gct ata tgc aa-3′ (SEQ ID NO: 17) and 5′-tag aca aac tat gaa agt t-3′ (SEQ ID NO: 18)]; and a primer set that is specific to fimbrial fimA genes from Porphyromonas gulae strains of group C [5′-cga tta tga cct tgt cgg taa gag ctt gga-3′ (SEQ ID NO: 13) and 5′-tgt ggc ttc gtt gtc gca gaa tcc ggc atg-3′ (SEQ ID NO: 14)] or [5′-gat ttg ctg ctc ttg cta tga cag ctt gta-3′ (SEQ ID NO: 19) and 5′-ttt agt cgt ttg acg ggt cga tta cca agt-3′ (SEQ ID NO: 20)]; whereby fimbrial types are detected.

14. A kit for identifying the groups of Porphyromonas gulae strains, which comprises primer sets for classifying Porphyromonas gulae FimA (fimbrillin) protein into three groups, i.e., groups A to C.

15. The kit according to claim 14, which comprises the primer sets:
a primer set that is specific to fimbrial fimA genes from Porphyromonas gulae strains of group A [5′-tga gaa tat caa atg tgg tgc agg ctc acg-3′ (SEQ ID NO: 9) and 5′-ctt gcc tgc ctt caa aac gat tgc ttt tgg-3′ (SEQ ID NO: 10)] or [5′-ttc ata cgt cga cga ctg cg-3′ (SEQ ID NO: 15) and 5′-ttg agg gtt gat tac caa gt-3′ (SEQ ID NO: 16)];
a primer set that is specific to fimbrial fimA genes from Porphyromonas gulae strains of group B [5′-taa gat tga agt gaa gat gag cga ttc tta tgt-3′ (SEQ ID NO: 11) and 5′-att tcc tca gaa ctc aaa gga gta cca tca-3′ (SEQ ID NO: 12)] or [5′-aac tac gac gct ata tgc aa-3′ (SEQ ID NO: 17) and 5′-tag aca aac tat gaa agt t-3′ (SEQ ID NO: 18)]; and
a primer set that is specific to fimbrial fimA genes from Porphyromonas gulae strains of group C [5′-cga tta tga cct tgt cgg taa gag ctt gga-3′ (SEQ ID NO: 13) and 5′-tgt ggc ttc gtt gtc gca gaa tcc ggc atg-3′ (SEQ ID NO: 14)] or [5′-gat ttg ctg ctc ttg cta tga cag ctt gta-3′ (SEQ ID NO: 19) and 5′-ttt agt cgt ttg acg ggt cga tta cca agt-3′ (SEQ ID NO: 20)].

16. A protein comprising the partial peptide according to claim 2.

17. An antibody which binds to the partial peptide according to claim 2 but not to a protein comprising the amino acid sequence of SEQ ID NO: 2 or 4.

18. An antibody which binds to the protein according to claim 4 but not to a protein comprising the amino acid sequence of SEQ ID NO: 2 or 4.

19. An antibody which binds to the protein according to claim 5 but not to a protein comprising the amino acid sequence of SEQ ID NO: 2 or 4.

20. The process according to claim 10, wherein the amino acid sequences of the FimA protein are specified by sequencing the entire nucleotide sequences of genes encoding the FimA protein from the Porphyromonas gulae strains.


SEQUENCE LISTING (22 sequences)

SEQ ID NO: 1 - DNA, 1327 bp, Porphyromonas gulae
  CDS (118)..(1272)
cctctatagctctatctcctttcatacgtcgacgactgcgacactatatgacaaggacaatctctaaatcgaaaaagatcctaataaaacaatattcacttttaaaacaaaaacgagatgaaaaagactaagtttttcttgttgggacttgctgcccttgctatgacagcttgtaacaaagacaacgaagcagaacccgttgtagaaggtaacgctaccattagcgtagtattgaagaccagcaatccgaatcgtgctttcggggttgcagatgacgaagcaaaagtggctaagttgaccgtaatggtttataatggagaacagcaggaagccatcgaatcagccgaaaatgcgactaagattgagaatatcaaatgtggtgcaggctcacgtacgctggtcgtaatggccaatacgggaacaatggatctgactggcaagactcttgcagatgtaaaagcattgacaactgaactgactgcagaaaaccaagaggctgcaggtttgatcatgacggcagagccaaaagcaatcgttttgaaggcaggcaagaactatattggatacaatggagccggagagggcaaccacattgagaatgatcctcttgagatcaagcgtgtacatgctcgcatggctttcaccgaaattaaagtgcagatgagcgcagcctacgataacatttacacatttgctcctgaaaagatttatggtctcattgcaaagaagcaatctaatttgttcggggcaacactcgtgaatgcagacgctaattatctgacaggttctttgaccacatttaacggtgcttacacacctgccaactatgccaatgtgccttggctgagccgtaattacgttgcacctaccgccaatgctcctcagggcttctatgtattagaaaatgactactcagctaacggtgggactattcatccgacaatcctatgtgtttatggcaaacttcagaaaaacggagccgacctggcgggagccgatttagcagctgctcaggccgccaattgggtggacgcagaaggcaagacctattaccctgtattggtaaacttcaacagcaacaactatacttatgacaatggttatacgcctaagaataaaattgagcgtaaccataagtatgatattaagctgacaatcacagggcctggaacaaacaatcccgagaatcctatcacagagtctgctcacttgaacgtacagtgtactgtagctgagtgggttctcgttggtcagaatgctacttggtaatcaaccctcaaaccaataaaagaaaaaacttcatagttgtacatgattagaatggagt

SEQ ID NO: 2 - protein, 383 aa, Porphyromonas gulae
MKKTKFFLLGLAALAMTACNKDNEAEPVVEGNATISVVLKTSNPNRAFGVADDEAKVAKLTVMVYNGEQQEAIESAENATKIENIKCGAGSRTLVVMANTGTMDLTGKTLADVKALTTELTAENQEAAGLIMTAEPKAIVLKAGKNYIGYNGAGEGNHIENDPLEIKRVHARMAFTEIKVQMSAAYDNIYTFAPEKIYGLIAKKQSNLFGATLVNADANYLTGSLTTFNGAYTPANYANVPWLSRNYVAPTANAPQGFYVLENDYSANGGTIHPTILCVYGKLQKNGADLAGADLAAAQAANWVDAEGKTYYPVLVNFNSNNYTYDNGYTPKNKIERNHKYDIKLTITGPGTNNPENPITESAHLNVQCTVAEWVLVGQNATW

SEQ ID NO: 3 - DNA, 1276 bp, Porphyromonas gulae
  CDS (83)..(1246)
aactacgacgctatatgcaagacaatctctaaatcaaaaaagatcctaataaaacaatattcacttttaaaacaaaaacaagatgaaaaagactaagtttttcttgttgggacttgccgcccttgctatgacagcttgtaacaaagacaacgaagcagaacccattgtagaaggtaacgctaccattagcgtagtattgaagaccagcaatccgaatcgtgctttcggggttgcagatgacgaagcaaatgtggctaagttgaccgtaatggtttataatggagaacagcaggaagccatcgaatcagccgaaaatgcaattaaggttgagaacatcaaatgtggtgcaggctcacgtacgctggtcgtaatggccaatacgggtggaatggaattggctggcaagactcttgcagaggtaaaagcattgacaactgaactaactgcagaaaaccaagaggctacaggtttgatcatgacagcagagcctgttgacgtaatacttgtcgccggcaataaccattatggttatgatggaactcagggaggcaatcagatttcgcaaggtactcctcttgaaatcaaacgtgttcatgcccgtattgcgttcactaagattgaagtgaagatgagcgattcttatgtgaacaaatacaactttacccccgaaaacatctatgcacttgtggctaagaagaagtctaatctattcggtacttcattggcaaatagtgatgatgcttatttgaccggttctttgacgactttcaacggtgcttatacccctgcaaactatactcatgtcgcctggttgggaagaggctacacagcgccttccaatgatgctccacaaggtttctatgttttggagagtgcatacgctcagaatgcaggtctacgtcctaccattctatgtgtaaagggtaagctgacaaagcatgatggtactcctttgagttctgaggaaatgacagctgcattcaatgccggctggattgttgcaaacaatgatcctacgacctattatcctgtattagtgaactttgagagcaataattacacctacacaggtgatgctgttgagaaagggaaaatcgttcgtaaccacaagtttgacatcaatctgacgatcaccggtcctggtacgaataatcctgaaaaccccattacagagtctgctaacctcaacgttaattgtgtggttgctgcgtggaaaggtgttgtacaaaatgttatttggtaatcagctcatcaaagaactttcatagtttgtcta

SEQ ID NO: 4 - protein, 386 aa, Porphyromonas gulae
MKKTKFFLLGLAALAMTACNKDNEAEPIVEGNATISVVLKTSNPNRAFGVADDEANVAKLTVMVYNGEQQEAIESAENAIKVENIKCGAGSRTLVVMANTGGMELAGKTLAEVKALTTELTAENQEATGLIMTAEPVDVILVAGNNHYGYDGTQGGNQISQGTPLEIKRVHARIAFTKIEVKMSDSYVNKYNFTPENIYALVAKKKSNLFGTSLANSDDAYLTGSLTTFNGAYTPANYTHVAWLGRGYTAPSNDAPQGFYVLESAYAQNAGLRPTILCVKGKLTKHDGTPLSSEEMTAAFNAGWIVANNDPTTYYPVLVNFESNNYTYTGDAVEKGKIVRNHKFDINLTITGPGTNNPENPITESANLNVNCVVAAWKGVVQNVIW

SEQ ID NO: 5 - DNA, 1294 bp, Porphyromonas gulae
  CDS (107)..(1273)
aatctgaacgaactgcgacgctatatgcaggacaatctctaagaaagaactgctatagatcgtatgaatcatacatatcaatattcacttttaaaacaaaaaagagatgaaaaaaacaaagtttttcttgttgggacttgctgcccttgcgatgacagcttgtaacaaagacaacgaagcagagcccattgtggaaacagacgctactgttagtttcataattaagagcggagagggtcgcgctgtaggcgatggtcttgcagatgccaagatcacaaaactcaccgccatggtctatgctggtcaaattcaagaaggtattaagacagtggaagaggctggcggggttcttaaagtggaaggaatccagtgtaaatcaggagccaaccgtgtcctcgtcattgtggctaatcacgattatgaccttgtcggtaagagcttggatcaggttgaggctttgacaacttctttgacagctgaaaaccaaaatgcccaaaacttgattatgacaggcaaatctgcagcttttacaattaagccgggctccaaccactatggctatcctgatgggactgcatccgacaaccttgtttctgctggtgctcctcttgccgttactcgcgtgcatgccggtatctcattcgcaggagtagaggtaaatatggctactcagtatcaaaactactattcttttaacccagccgatgctaagatcgcagcccttgtagcaaagaaagactctaagattttcggcgatcctttgttctcagactctaaggcatatttgtatggggttcaaacgcctgcaggcctttacactcccgatgctaccggtgaaacatacgagttggaagcgtctttgaatatgaactatgctgaaggtgccggcttctatgtactggaaagcaaatatgatgtaaccaacgagcttcgtcccacgatcctttgtatctatgggaagctgctcgataaggacggcaaccctctcacgggacaagccttgacggatgctatccatgccggattctgcgacaacgaagccacaacttactatccggtattggtgaactatgatggcaatggctacatctattctggtaatattacccaaggacaaaacaaaatcgttcgcaacaaccactacaagattacgctgaatatcaccggccccggtacggatactcctgaaaatcctcaaccggtacaagccaacctgaatgttacttgcgaagttacaccttgggttgttgttaatcaggctgctacttggtaatcgacccgtcaaacgactaaa

SEQ ID NO: 6 - protein, 388 aa, Porphyromonas gulae
MKKTKFFLLGLAALAMTACNKDNEAEPIVETDATVSFIIKSGEGRAVGDGLADAKITKLTAMVYAGQIQEGIKTVEEAGGVLKVEGIQCKSGANRVLVIVANHDYDLVGKSLDQVEALTTSLTAENQNAQNLIMTGKSAAFTIKPGSNHYGYPDGTASDNLVSAGAPLAVTRVHAGISFAGVEVNMATQYQNYYSFNPADAKIAALVAKKDSKIFGDPLFSDSKAYLYGVQTPAGLYTPDATGETYELEASLNMNYAEGAGFYVLESKYDVTNELRPTILCIYGKLLDKDGNPLTGQALTDAIHAGFCDNEATTYYPVLVNYDGNGYIYSGNITQGQNKIVRNNHYKITLNITGPGTDTPENPQPVQANLNVTCEVTPWVVVNQAATW

SEQ ID NO: 7 - DNA, 20 bp, Artificial
  Forward primer specific for Porphyromonas gulae.
ttgcttggttgcatgatcgg

SEQ ID NO: 8 - DNA, 24 bp, Artificial
  Reverse primer specific for Porphyromonas gulae.
gcttattcttacggtacattcaca

SEQ ID NO: 9 - DNA, 30 bp, Artificial
  Forward primer for amplifying group A fimA gene.
tgagaatatcaaatgtggtgcaggctcacg

SEQ ID NO: 10 - DNA, 30 bp, Artificial
  Reverse primer for amplifying group A fimA gene.
cttgcctgccttcaaaacgattgcttttgg

SEQ ID NO: 11 - DNA, 33 bp, Artificial
  Forward primer for amplifying group B fimA gene.
taagattgaagtgaagatgagcgattcttatgt

SEQ ID NO: 12 - DNA, 30 bp, Artificial
  Reverse primer for amplifying group B fimA gene.
atttcctcagaactcaaaggagtaccatca

SEQ ID NO: 13 - DNA, 30 bp, Artificial
  Forward primer for amplifying group C fimA gene.
cgattatgaccttgtcggtaagagcttgga

SEQ ID NO: 14 - DNA, 30 bp, Artificial
  Reverse primer for amplifying group C fimA gene.
tgtggcttcgttgtcgcagaatccggcatg

SEQ ID NO: 15 - DNA, 20 bp, Artificial
  Forward primer for amplifying group A fimA gene.
ttcatacgtcgacgactgcg

SEQ ID NO: 16 - DNA, 20 bp, Artificial
  Reverse primer for amplifying group A fimA gene.
ttgagggttgattaccaagt

SEQ ID NO: 17 - DNA, 20 bp, Artificial
  Forward primer for amplifying group B fimA gene.
aactacgacgctatatgcaa

SEQ ID NO: 18 - DNA, 19 bp, Artificial
  Reverse primer for amplifying group B fimA gene.
tagacaaactatgaaagtt

SEQ ID NO: 19 - DNA, 30 bp, Artificial
  Forward primer for amplifying group C fimA gene.
gatttgctgctcttgctatgacagcttgta

SEQ ID NO: 20 - DNA, 30 bp, Artificial
  Reverse primer for amplifying group C fimA gene.
tttagtcgtttgacgggtcgattaccaagt

SEQ ID NO: 21 - DNA, 631 bp, Porphyromonas gulae
  CDS (2)..(631)
cgattatgaccttgtcggtaagagcttggatcaggttgaggctttgacaacttctttgacagctgaaaaccaaaatgcccaaaacttgattatgacaggcaaatctgcagcttttacaattaagccgggctccaaccactatggctatcctgatgggactgcatccgacaaccttgtttctgctggtgctcctcttgccgttactcgcgtgcatgccggtatctcattcgcaggagtagaggtaaatatggctactcagtatcaaaactactattcttttaacccagccgatgctaagatcgcagcccttgtagcaaagaaagactctaagattttcggcgatcctttgttctcagactctaaggcatatttgtatggggttcaaacgcctgcaggcctttacactcccgatgctaccggtgaaacatacgagttggaagcgtctttgaatatgaactatgctgaaggtgccggcttctatgtactggaaagcaaatatgatgtaaccaacgagcttcgtcccacgatcctttgtatctatgggaagctgctcgataaggacggcaaccctctcacgggacaagccttgacggatgctatccatgccggattctgcgacaacgaagccaca

SEQ ID NO: 22 - protein, 210 aa, Porphyromonas gulae
DYDLVGKSLDQVEALTTSLTAENQNAQNLIMTGKSAAFTIKPGSNHYGYPDGTASDNLVSAGAPLAVTRVHAGISFAGVEVNMATQYQNYYSFNPADAKIAALVAKKDSKIFGDPLFSDSKAYLYGVQTPAGLYTPDATGETYELEASLNMNYAEGAGFYVLESKYDVTNELRPTILCIYGKLLDKDGNPLTGQALTDAIHAGFCDNEAT